(19) United States
(12) Patent Application Publication
(10) Pub. No.: US 2015/0276725 A1
(43) Pub. Date: Oct. 1, 2015

(54) CHEMILUMINESCENT APTASENSORS

(71) Applicant: LUMINESCENT MD, LLC, Gaithersburg, MD (US)

(72) Inventor: Ji Hoon Lee, Gaithersburg, MD (US)

(73) Assignee: LUMINESCENT MD, LLC, Gaithersburg, MD (US)

(21) Appl. No.: 14/434,610

(22) PCT Filed: Oct. 10, 2013
(86) PCT No.: PCT/US2013/064252

§ 371 Date: Apr. 9, 2015

Related U.S. Application Data
(60) Provisional application No. 61/712,072, filed on Oct. 10, 2012.

(51) Int. Cl. G01N 33/53; G01N 33/543; G01N 33/58

(52) U.S. Cl. CPC G01N 33/5308; G01N 33/582; G01N 33/54313; G01N 2458/30

(57) ABSTRACT

A chemiluminescent immunoassay for sensing an analyte in a sample includes an oligonucleotide, a buffer solution, a chemiluminescent reagent, and a micro-particle or a nano-particle. An analyte in a sample is detected by conjugating an oligonucleotide with (i) a fluorescent dye or a fluorescent polystyrene bead and (ii) a micro-particle or a nano-particle; mixing the conjugated oligonucleotide and a chemiluminescent reagent; and measuring light intensity generated as a result of mixing the conjugated oligonucleotide and chemiluminescent reagent. The oligonucleotide advantageously captures and detects the analyte without the requirement of an anti-body. The micro-particle or nano-particle removes excess oligonucleotide without the requirement of washing.

### FIELD OF INVENTION

This invention relates to a chemiluminescence system capable of sensing biomarkers or toxic materials bound with single strand DNA and RNA oligonucleotides.

### BACKGROUND

Since 1992, it has been well-known that single strand DNA (ssDNA) as well as RNA oligonucleotides, instead of antibody, could be used as a capture capable of binding biomarkers and toxic materials. Also, ssDNA and RNA oligonucleotides conjugated with various labels (e.g., fluorescent dyes, biotin, aminated and carbonated compounds) have been used in various detection methods. Thus, they can be applied like detection antibodies used in various immunoassays.

Using the advantages of ssDNA and RNA oligonucleotides, novel biosensors with 1,1′-oxalyldiimidazole (ODI) derivative chemiluminescence detection have been developed to rapidly quantify and monitor analytes such as biomarkers and toxic materials.

### BRIEF DESCRIPTION OF THE DRAWINGS

FIG. 1 shows π-π stacking interaction between ssDNA-conjugated TEX615 and PDIMFs;

FIG. 2 shows possible mechanisms capable of sensing V. parahaemolyticus using ODI-CL aptasensor using GO and ssDNA oligonucleotides;

FIG. 3 is a calibration curve for the quantification of V. parahaemolyticus using ODI-CL aptasensor with GO and ssDNA oligonucleotides;

FIG. 4 shows the effect of fluorescent dye labeled with ssDNA or RNA oligonucleotide in ODI-CL aptasensor using ssDNA or RNA oligonucleotides; and

FIG. 5 shows the effect of fluorescent dye coated on the surface of polystyrene bead in ODI-CL aptasensor using ssDNA or RNA oligonucleotides.

### DETAILED DESCRIPTION

The present invention is a biosensor with 1,1′-oxalyldiimidazole (ODI) derivative detection capable of sensing analytes (e.g., biomarkers, toxic materials) bound with ssDNA or RNA oligonucleotides, which are conjugated with various labels (e.g., fluorescent dye, biotin, aminated and carbonated compounds).

Oligonucleotides synthesized to use in developing biosensors capable of various analytes are single strand DNA (ssDNA) and RNA oligonucleotides.

Amino or carboxyl magnetic beads used to immobilize ssDNA oligonucleotides or capture antibody are ferromagnetic and paramagnetic.

Fluorescent dyes labeled with ssDNA or RNA oligonucleotides are Cy3, CY3.5, Cy5, Cy5.5, Cy7, Fluorescein, 6-FAM, Perylene, Rhodamine Green, Rhodamine Red, ROX, TAMRA, Texas Red, and TEX615.

Fluorescent polystyrene beads labeled with ssDNA or RNA oligonucleotides.

Fluorescent dye coated on the surface of polystyrene bead is coumarin, fluorescein, rhodamine, or phycoerithrin

Nanoparticles (e.g.,), capable of weakly binding with ssDNA or RNA oligonucleotides due to the π-π stacking interaction between nanoparticles and oligonucleotides, are single- and multi-walled carbon nanotubes, graphene, graphene oxide, gold and silver nano-particles.

A microparticle capable of weakly binding with ssDNA or RNA oligonucleotides is 3,4,9,10-perylenetetracarboxylic diimide microfibers.

Chemiluminescence reagents used in ODI derivative CL reaction are 1,1′-oxalyldiimidazole (ODI), 1,1′-oxalydi-2-ethyl-imidazole (OD2EI), 1,1′-oxalyl-2-methyl-imidazole (OD2MI), and 1,1′-oxalyl-4-methyl-imidazole (OD4MI).

Substrates used in biosensors with 1,1′-oxalyldiimidazole (ODI) derivative chemiluminescence detection and streptavidin-conjugated HRP are Amplex Red, 2,3-diaminophenazine.

Substrates used in biosensors with 1,1′-oxalyldiimidazole (ODI) derivative chemiluminescence detection and streptavidin-conjugated ALP are fluorescein diphosphate (FDP), 4-methyl umbelliferyl phosphate (MUP), 3-O-methyl fluorescein phosphate.

### EXAMPLES

### Example 1

### Competitive Binding of Analytes and Oligonucleotides to Micro- or Nano-Particles

A. Interaction of oligonucleotides and micro- or nano-particles Preparation

0.5 μM of single strand DNA (ssDNA) oligonucleotides conjugated with TEX615, capable of binding to vibrio parahaemolyticus, or Ochratoxin A (OTA) was prepared in PBS (10 mM sodium phosphate, 137 mM sodium chloride, 2.7 mM potassium chloride, pH 7.4).

0.5 μM of RNA oligonucleotides conjugated with TEX615, capable of binding to E. Coli O157:H7, was prepared in PBS (10 mM sodium phosphate, 137 mM sodium chloride, 2.7 mM potassium chloride, pH 7.4).

0.01 mg/ml of 3,4,9,10-perylenetetracarboxylic diimide fibers (PDIMFs), as a micro-particle, was prepared in PBS (10 mM sodium phosphate, 137 mM sodium chloride, 2.7 mM potassium chloride, pH 7.4).

Single-walled carbon nanotubes (0.04 mg/ml) and multi-walled carbon nanotubes (0.04 mg/ml) were prepared in PBS (10 mM sodium phosphate, 137 mM sodium chloride, 2.7 mM potassium chloride, pH 7.4).

Graphene oxide (0.04 mg/ml) and grapheme (0.04 mg/ml) were prepared in PBS (10 mM sodium phosphate, 137 mM sodium chloride, 2.7 mM potassium chloride, pH 7.4).

Gold (10 ppm) and silver (10 ppm) nano-particles were prepared in PBS (10 mM sodium phosphate, 137 mM sodium chloride, 2.7 mM potassium chloride, pH 7.4).

1,1′-Oxalyldi-4-methyl-imidazole (OD4MI), one of ODI derivatives formed from the reaction between 5.0 μM TCPO, and 10.0 μM 4-Methylimidazole (4MImH) in Ethyl acetate. 100 mM H₂O₂ was prepared in Isopropyl alcohol.

### Procedure

1. ssDNA or RNA oligonucleotides (0.5 ml) were mixed with micro- or nano-particles (0.5 ml) in a 1.5 ml-centrifuge tube.
2. The mixture in the centrifuge tube was incubated at room temperature for 30 minutes.
3. 10 μl of mixture was inserted into the assigned test tube (12×75 mm)
4. Insert the test tube into a luminometer into LB 9507 Luminometer with two dispensers (Berthold Technologies).
5. CL emitted when OD4MI and H₂O₂ are added into the test tube through two dispensers was measured.

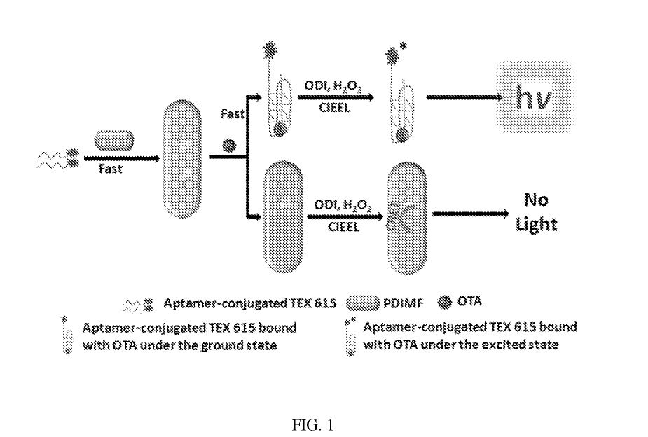

ssDNA oligonucleotides conjugated with TEX615 in the absence of micro- or nano-particles were emitted strong light when OD4MI and H₂O₂ were injected into the test tube. However, CL emission of ssDNA oligonucleotides conjugated with TEX615 in the presence of micro- or nano-particles was not measured or was detected weak signal. This is because ssDNA oligonucleotides conjugated with TEX615 were bound with micro- or nano-particle, due to the π-π interaction between ssDNA oligonucleotides and micro- or nano-particle. FIG. 1 shows that ssDNA oligonucleotides immobilized on the surface of PDIMFs cannot emit light due to the chemiluminescent resonance energy transfer (CRET) between TEX615 labeled with ssDNA and PDIMF in ODI CL reaction.

Due to the π-π stacking interaction between RNA oligonucleotides and micro- or nano-particle, also, CL emission of RNA oligonucleotides conjugated with TEX615 in the presence of micro- or nano-particles wasn't measured or was detected weak signal even though RNA oligonucleotides conjugated with TEX615 in the absence of micro- or nano-particles were emitted strong light when OD4MI and H₂O₂ were injected into the test tube. Table 1 shows that RNA-conjugated TEX615 immobilized on the surface of grapheme oxide cannot emit light due to CRET between RNA-conjugated TWX615 and carbon nanotube (CNT). Relative CL intensity measured in the presence of CNT was similar to the background measured in the absence of RNA-conjugated TEX615.

TABLE 1
ODI-CL emission of RNA-conjugated TEX615
in the absence and presence of CNT

| | Relative CL intensity in the | Relative CL intensity in the |
|---|---|---|
| | absence of CNT | presence of CNT |
| RNA-conju- | 2.2 × 10⁵ | 3.1 × 10² |
| gated TEX615 | | |

B. Interaction of vibrio parahaemolyticus and ssDNA oligonucleotides in the presence of grapheme oxide

### Preparation

Various concentrations of vibrio parahaemolyticus were prepared in Tris-EDTA under various pH (e.g., 7, 7.5, 8, 8.5).

0.5 μM of ssDNA oligonucleotides conjugated with TEX615, capable of binding with vibrio parahaemolyticus, was prepared in PBS (10 mM sodium phosphate, 137 mM sodium chloride, 2.7 mM potassium chloride, pH 7.4).

Graphene oxide (0.04 mg/ml, GO) was prepared in PBS (10 mM sodium phosphate, 137 mM sodium chloride, 2.7 mM potassium chloride, pH 7.4).

1,1′-Oxalyldi-4-methyl-imidazole (OD4MI), one of ODI derivatives, formed from the reaction between 5.0 μM TCPO and 10.0 μM 4-Methylimidazole (4MImH) in Ethyl acetate. 100 mM H₂O₂ was prepared in Isopropyl alcohol.

1.0 mM TCPO and 10 μM imidazole were prepared in Ethyl acetate. 100 mM H₂O₂ was prepared in Isopropyl alcohol.

### Procedure

1. Vibrio parahaemolyticus (0.3 ml) was mixed with grapheme oxide (0.3 ml) in a 1.5 ml centrifuge tube.
2. ssDNA oligonucleotides conjugated with TEX615 (0.3 ml) were added in the centrifuge tube containing vibrio parahaemolyticus and grphene oxide.
3. The mixture in the centrifuge tube was incubated for 10 minutes under various temperatures (e.g., 4, 21, 37° C.).
4. 10 μl of mixture was inserted into the assigned test tube (12×75 mm)
5. Insert the test tube into a luminometer into LB 9507 Luminometer with two dispensers (Berthold Technologies).
6. Light emitted in the test tube with the addition of CL reagents for ODI-CL through two dispensers of LB 9507 Luminometer was measured.

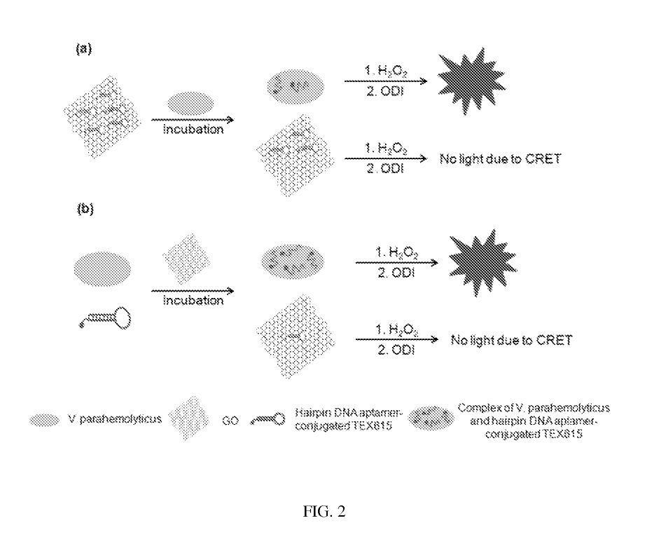

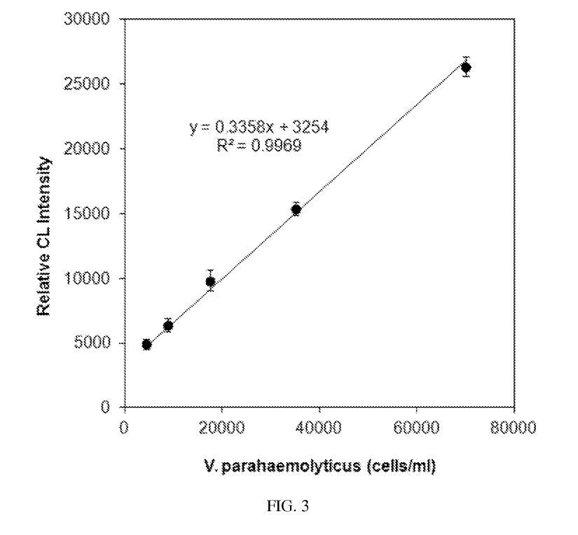

Relative CL intensity emitted from vibrio parahaemolyticus (V. parahaemolyticus) bound with ssDNA oligonucleotides conjugated TEX615 in the presence of grapheme oxide was measured using ODI-CL detection. Relative CL intensity was dependent on the concentration of vibrio parahaemolyticus. FIG. 2 shows the possible mechanisms capable of sensing vibrio parahaemolyticus using ODI-CL aptasensor using ssDNA oligos. FIG. 3 shows that ODI-CL aptasensor can quantify trace levels of V. parahaemolyticus.

C. Interaction of E. Coli O157:H7 and RNA Oligos in the Presence of Grapheme Oxide Preparation

Various concentrations of E. Coli O157:H7 were prepared in Tris-EDTA under various pH (e.g., 7, 7.5, 8, 8.5).

0.5 μM of RNA oligonucleotides conjugated with TEX615, capable of binding with vibrio parahaemolyticus, was prepared in PBS (10 mM sodium phosphate, 137 mM sodium chloride, 2.7 mM potassium chloride, pH 7.4).

Graphene oxide (0.04 mg/ml) was prepared in PBS (10 mM sodium phosphate, 137 mM sodium chloride, 2.7 mM potassium chloride, pH 7.4).

1,1′-Oxalyldi-4-methyl-imidazole (OD4MI), one of ODI derivatives, formed from the reaction between 5.0 μM TCPO and 10.0 μM 4-Methylimidazole (4MImH) in Ethyl acetate. 100 mM H₂O₂ was prepared in Isopropyl alcohol.

### Procedure

1. E. Coli O157:H7 (0.3 ml) was mixed with grapheme oxide (0.3 ml) in a 1.5 ml centrifuge tube.
2. RNA oligonucleotides conjugated with TEX615 (0.3 ml) were added in the centrifuge tube containing vibrio parahaemolyticus and grphene oxide.
3. The mixture in the centrifuge tube was incubated for 10 minutes under various temperatures (e.g., 4, 21, 37° C.).
4. 10 μl of mixture was inserted into the assigned test tube (12×75 mm)
5. Insert the test tube into a luminometer into LB 9507 Luminometer with two dispensers (Berthold Technologies).
6. Light emitted in the test tube with the addition of CL reagents for conventional peroxyoxalate, ODI, or ODB CL through two dispensers of LB 9507 Luminometer was measured.

Relative CL intensity emitted from E. Coli O157:H7 bound with RNA oligonucleotides conjugated TEX615 in the presence of grapheme oxide was measured using ODI-CL detection. Relative CL intensity was dependent on the concentration of E. Coli O157:H7. ODI CL detection was very accurate, precise, sensitive and reproducible as shown in Table 2.

TABLE 2
Accuracy, precision, and Recovery of biosensor (n = 5).

| | | | | Accuracy | Precision | Recovery |
|---|---|---|---|---|---|---|
| Sample 1^{a} | Sample 2^{a} | Calculated^{a} | Result^{a} | (%) | (%) | (%) |
| 10,000 | 120,000 | 65,000 | 62,224 | 4.3 | 5.4 | 95.7 |
| 17,500 | 70,000 | 43,750 | 45,288 | 3.5 | 5.0 | 103.5 |
| 35,000 | 70,000 | 52,500 | 51,383 | 2.1 | 3.9 | 97.9 |

^{a}cell/ml of E. Coli O157:H7

### Example 2

### Effect of Fluorescent Dyes Labeled with ODI-CL Aptasensor Using ssDNA or RNA Oligos

0.5 μM ssDNA oligonucleotides conjugated with a fluorescent dye (e.g., Cy3, CY3.5, Cy5, Cy5.5, Cy7, Fluorescein, 6-FAM, Perylene, Rhodamine Green, Rhodamine Red, ROX, TAMRA, Texas Red, TEX615), capable of binding with vibrio parahaemolyticus, were prepared in Tris-EDTA buffer (pH 7.5).

1,1′-Oxalyldi-4-methyl-imidazole (OD4MI), one of ODI derivatives, formed from the reaction between 5.0 μM TCPO and 10.0 μM 4-Methylimidazole (4MImH) in Ethyl acetate. 100 mM H₂O₂ was prepared in Isopropyl alcohol.

### Procedure

1. 10 μl of ssDNA oligonucleotides conjugated with fluorescent dye was inserted into the assigned test tube (12×75 mm)
2. Insert the test tube into a luminometer into LB 9507 Luminometer with two dispensers (Berthold Technologies).
3. Light emitted in the test tube with the addition of CL reagents for conventional peroxyoxalate, ODI, or ODB CL through two dispensers of LB 9507 Luminometer was measured.

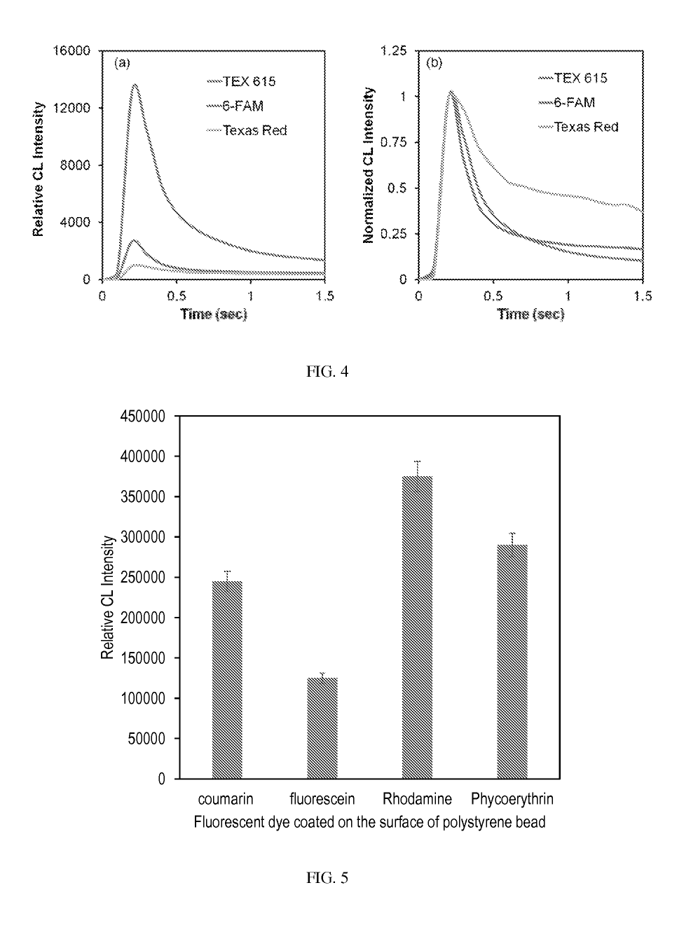

Using three different CL detection methods, CL mitted from ssDNA oligonucleotides conjugated with fluorescent dye was measured. As shown in FIG. 4, relative CL intensity was dependent on the chemical and physical properties of fluorescent dye labeled with ssDNA or RNA oligonucleotides. However, all fluorescent dyes can be labeled with ssDNA oligonucleotides to quantify trace levels of biomarkers and toxic materials.

### Example 3

### Interaction of ssDNA oligos conjugated with fluorescent polystyrene bead and graphene oxide

### Preparation

0.1 μM of ssDNA oligonucleotides conjugated with fluorescent polystyrene bead was prepared in PBS (10 mM sodium phosphate, 137 mM sodium chloride, 2.7 mM potassium chloride, pH 7.4).

Graphene oxide (0.04 mg/ml) was prepared in PBS (10 mM sodium phosphate, 137 mM sodium chloride, 2.7 mM potassium chloride, pH 7.4).

1,1′-Oxalyldi-4-methyl-imidazole (OD4MI), one of ODI derivatives, formed from the reaction between 5.0 μM TCPO and 10.0 μM 4-Methylimidazole (4MImH) in Ethyl acetate. 100 mM H₂O₂ was prepared in Isopropyl alcohol.

### Procedure

1. The mixture of ssDNA-conjugated biotin (200 nM) and streptavidin-conjugated fluorescent polystyrene (0.01% (w/v)) was incubated for 30 minutes.
2. ssDNA oligos conjugated with fluorescent polystyrene bead (0.5 ml) were mixed with graphene oxide (0.5 ml) in a 1.5 ml-centrifuge tube.
3. The mixture in the centrifuge tube was incubated at room temperature for 30 minutes.
4. 10 μl of mixture was inserted into the assigned test tube (12×75 mm)
5. Insert the test tube into a luminometer into LB 9507 Luminometer with two dispensers (Berthold Technologies).
6. CL emitted when OD4MI and H₂O₂ are added into the test tube through two dispensers was measured.

ssDNA oligonucleotides conjugated with fluorescent polystyrene bead in the absence of micro- or nano-particles were emitted strong light when OD4MI and H₂O₂ were injected into the test tube. However, CL emission of ssDNA oligonucleotides conjugated with fluorescent polystyrene bead in the presence of micro- or nano-particles was not measured or a weak signal was detected. This is because ssDNA oligonucleotide-conjugated fluorescent polystyrene bead immobilized on the surface of micro- or nano-particle, due to the π-π interaction between ssDNA oligonucleotides and micro- or nano-particle, cannot emit light based on the principle of CRET between fluorescent polystyrene bead-labeled ssDNA and micro- or nano-particle. In addition, as shown in FIG. 5, relative CL intensity of ODI-CL aptasensor is dependent on the property of fluorescent dye coated on the surface of polystyrene bead.

### CONCLUSION

While various embodiments of the present invention have been described above, it should be understood that they have been presented by way of example only, and not limitation. It will be understood by those skilled in the art that various changes in form and details may be made therein without departing from the spirit and scope of the invention. Thus, the breadth and scope of the invention should not be limited by any of the above-described exemplary embodiments.

What is claimed is:

1. A chemiluminescent immunoassay for sensing an analyte in a sample, comprising: an oligonucleotide, a buffer solution, a chemiluminescent reagent, and a micro-particle or a nano-particle.

2. The immunoassay of claim 1, wherein the oligonucleotide is a single-stranded DNA or RNA nucleotide.

3. The immunoassay of claim 1, wherein the buffer solution is selected from the group consisting of phosphate buffered saline (PBS), tris-buffered saline (TBS), and tris-EDTA (TE) buffer solution.

4. The immunoassay of claim 1, wherein the micro-particle is 3,4,9,10-perylenetetracarboxylic diimide microfibers.

5. The immunoassay of claim 1, wherein the nano-particle is selected from the group consisting of single-walled and multi-walled carbon nanotube, graphene, graphene oxide, gold nano-particle and silver nano-particle.

6. The immunoassay of claim 1, wherein the chemiluminescent reagent comprises a 1,1′-oxalyldiimidazole (ODI) derivatives.

7. The immunoassay of claim 6, wherein the 1,1′-oxalyldiimidazole (ODI) derivative is selected from the group consisting of 1,1′-Oxalyldiimidazole; 1,1′-Oxalyldi-2-methyl-imidazole (OD2MI); 1,1′-Oxalyldi-2-ethyl-imidazole (OD2EI); and 2,1′-Oxalyldi-4-methyl-imidazole (OD4MI).

8. The immunoassay of claim 1, further comprising a fluorescent dye.

9. The immunoassay of claim 8, wherein the fluorescent dye is selected from the group consisting of: Cy3, CY3.5, Cy5, Cy5.5, Cy7, Fluorescein, 6-FAM, Perylene, Rhodamine Green, Rhodamine Red, ROX, TAMRA, Texas Red, and TEX615.

10. The immunoassay of claim 1, further comprising a fluorescent polystyrene bead.

11. The immunoassay of claim 10, wherein the fluorescent polystyrene bead is a micro-bead or a nano-bead.

12. The immunoassay of claim 10, wherein the fluorescent polystyrene bead is selected from the group consisting of coumarin-stained, fluorescein-stained, rhodamine-stained, and phycoerithrin-stained polystyrene beads.

13. A method for sensing an analyte in a sample, comprising:
conjugating an oligonucleotide with (i) a fluorescent dye or a fluorescent polystyrene bead and (ii) a micro-particle or a nano-particle;
mixing the conjugated oligonucleotide from said conjugating step and a chemiluminescent reagent; and
measuring light intensity generated as a result of said mixing step.

14. The method of claim 13, wherein the oligonucleotide comprises a single-stranded DNA (ssDNA) or RNA oligonucleotide.

15. The method of claim 13, wherein the chemiluminescent reagent comprises a 1,1′-oxalyldiimidazole (ODI) derivative.

16. The method of claim 15, wherein the 1,1′-oxalyldiimidazole (ODI) derivative is 1,1′-Oxalyldiimidazole; 1,1′-Oxalyldi-2-methyl-imidazole (OD2MI); 1,1′-Oxalyldi-2-ethyl-imidazole (OD2EI); or 2,1′-Oxalyldi-4-methyl-imidazole (OD4MI).

17. The method of claim 13, wherein the fluorescent dye is selected from the group consisting of Cy3, CY3.5, Cy5, Cy5.5, Cy7, Fluorescein, 6-FAM, Perylene, Rhodamine Green, Rhodamine Red, ROX, TAMRA, Texas Red, and TEX615.

18. The method of claim 13, wherein fluorescent polystyrene bead comprises a fluorescent polystyrene micro-bead or a fluorescent polystyrene nano-bead, and further wherein the fluorescent polystyrene bead is selected from the group consisting of coumarin-stained, fluorescein-stained, rhodamine-stained, and phycoerithrin-stained polystyrene beads.

19. The method of claim 13, wherein the micro-particle is 3,4,9,10-perylenetetracarboxylic diimide microfibers.

20. The method of claim 13, wherein the nano-particle is selected from the group consisting of single-walled and multi-walled carbon nanotube, graphene, graphene oxide, gold nano-particle and silver nano-particles.
